(19) United States
(12) Patent Application Publication
(10) Pub. No.: US 2022/0325271 A1
(43) Pub. Date: Oct. 13, 2022

(54) DROPLET-BASED SINGLE EXTRACELLULAR VESICLE SEQUENCING

(71) Applicants: The General Hospital Corporation, Boston, MA (US); President and Fellows of Harvard College, Cambridge, MA (US)

(72) Inventors: Jina Ko, Boston, MA (US); Ralph Weissleder, Peabody, MA (US); Yongcheng Wang, Brookline, MA (US); David A. Weitz, Cambridge, MA (US)

(21) Appl. No.: 17/765,145

(22) PCT Filed: Sep. 28, 2020
(86) PCT No.: PCT/US2020/053016

§ 371 Date: Mar. 30, 2022

Related U.S. Application Data
(60) Provisional application No. 62/908,432, filed on Sep. 30, 2019.

(51) Int. Cl. C12N 15/10; C40B 50/06; G01N 33/544; G01N 33/574

(52) U.S. Cl. CPC C12N 15/1003; C12N 15/1096; C40B 50/06; G01N 33/544; G01N 33/57492

(57) ABSTRACT

Described herein are methods, uses, and kits for droplet-based single cell sequencing of nucleic acids from extracellular vesicles. Specifically, the disclosure provides methods of analyzing protein compositions from individual extracellular vesicles (EVs) from biological samples including pluralities of EVs, the methods comprising labeling the EVs with antibody-DNA conjugates; encapsulating the labeled EVs, barcoded beads, and an extension reagent mix into droplets; within one or more of the droplets, hybridizing the antibody-DNA conjugates with a hybridization region in the barcoded beads; generating RNA from the DNA; synthesizing cDNA from the RNA; amplifying and sequencing the cDNA from one or more individual EVs from the biological sample; and analyzing the sequence of the cDNA from individual EVs to define their protein composition.

### CLAIM OF PRIORITY

This application claims the benefit of U.S. Patent Application Ser. No. 62/908,432, filed on Sep. 30, 2019.

### TECHNICAL FIELD

This invention relates to sample analysis techniques, and more particularly to methods, systems, and kits for detecting and analyzing extracellular vesicles from disease-derived cells, to determine which therapies would be most effective in a specific patient.

### BACKGROUND

Proteins are the primary effectors of cellular function, including cellular metabolism, structural dynamics, and information processing.

Measuring protein expression and modification is thus important for obtaining an accurate snapshot of cell state and function. A common challenge when measuring proteins at the single-extracellular vesicle (EV) level is that EVs are thousand times smaller in size compared to that of their cells of origin. Additionally, most cell systems and cell-derived EVs are heterogeneous, containing massive numbers of molecularly distinct cargo. This underlying EV heterogeneity can have important consequences on the system as a whole, such as in development, the regulation of the immune system, cancer progression, and therapeutic response. To help understand these systems, high-throughput protein profiling in single EVs is necessary.

Profiling proteins in single EVs at high throughput requires methods that are sensitive and fast. Flow cytometry with fluorescently-labeled antibodies has been a bedrock in biology for decades, because it can sensitively profile proteins in millions of single cells. Nevertheless, while these methods continue to improve in sensitivity and multiplexing for cells, they are still unable to characterize the entire proteome in single EVs due to their small sizes (30 nm-1 μm), which is below the limit of detection of the flow cytometry. Accordingly, new methods are needed.

### SUMMARY

The disclosure provides methods for droplet-based single EV profiling, uses of these methods, and kits for the use of these methods. Specifically, this disclosure is based, at least in part, on the discovery that droplet-based single EV profiling permits the detection and identification of diseased EV subtypes, which would otherwise be impossible to detect due to the co-presence of abundant normal EVs.

In a first aspect, this disclosure provides methods of analyzing protein compositions from individual extracellular vesicles (EVs) from biological samples including pluralities of EVs. The methods include isolating EVs from a biological sample; labeling the EVs with antibody-DNA conjugates; obtaining barcoded beads; encapsulating the labeled EVs, the barcoded beads, and an extension reagent mix into droplets; within one or more of the droplets, hybridizing a first hybridization region in the antibody-DNA conjugates with a second hybridization region in the barcoded beads to create hybridized DNA; extending the hybridized DNA within one or more of the droplets to generate extended DNA; generating RNA from the extended DNA; synthesizing cDNA from the RNA; amplifying and sequencing the cDNA from one or more individual EVs from the biological sample; and analyzing the sequence of the cDNA from individual EVs to define their protein composition.

In a second aspect, this disclosure provides methods of analyzing protein compositions from individual extracellular vesicles (EVs) from biological samples including pluralities of EVs. The methods include isolating EVs from a biological sample; labeling the EVs with antibody-DNA conjugates and purifying after labeling, each antibody-DNA conjugate including an antibody that binds to the EVs, a T7 promoter, a first barcode region, and a first hybridization region; obtaining barcoded beads each including beads conjugated to a unique molecular identifier (UMI) region, a second barcode region, and a second hybridization region that can hybridize to the first hybridization region; encapsulating the labeled EVs, the barcoded beads, and an extension reagent mix including deoxynucleotide triphosphates, a nonionic surfactant, a redox reagent, a DNA polymerase, and a Uracil-Specific Excision Reagent (USER) enzyme into droplets such that, on average, only one labeled EV and only one barcoded bead are encapsulated per droplet; within one or more of the droplets, hybridizing the first hybridization region attached to an EV with the second hybridization region attached to a barcoded bead, creating hybridized DNA; extending the hybridized DNA within one or more of the droplets to generate extended DNA; performing in vitro transcription (IVT) on the extended DNA, wherein RNA is generated from extended DNA by performing in vitro transcription (IVT) on the extended DNA; synthesizing cDNA from the RNA; amplifying and sequencing the cDNA from one or more individual EVs in the plurality of EVs in the biological sample; and analyzing the sequence of the cDNA from individual EVs to define their protein composition.

In some embodiments, the EVs are isolated from the biological sample by ultracentrifugation or size exclusion chromatography. In certain embodiments, the cDNA is amplified by conducting a polymerase chain reaction (PCR). Additionally, the antibodies that bind to the EVs can specifically bind to surface antigens present on tumor cells. In some variations, the methods further include characterizing the EVs after isolation from the biological sample. In some embodiments, the beads include one or more of a polyacrylamide, a cross-linked polyacrylamide, a polymer dissolvable on demand, a sepharose, or a hydrogel. In other embodiments, each of the regions of the DNA on the antibody-DNA conjugate includes 5-100 nucleotide bases. In some implementations, multiple antibody-DNA conjugates are used. In certain embodiments, each of the regions of the bead barcodes includes 5-50 nucleotide bases. In various implementations, the methods can also include, generating libraries of synthetic barcodes, wherein each barcode is different from each other barcode in at least one base, and wherein each barcode includes 5-20 nucleotide bases. In some examples, the EVs of the present methods are circulating EVs from a patient. Alternatively, the biological sample can be obtained from cultured cells. In certain embodiments, the biological sample is blood, saliva, urine, cerebrospinal fluid, cyst fluid, or a lavage. In some implementations, the EVs are semi-permeabilized to release intravesicular proteins. In various implementations, the EVs are microvesicles, exomeres, apoptotic bodies, oncosomes, endosomes, lysosomes, or mitochondria.

In another aspect, the disclosure provides methods of detecting and monitoring disease or disorder, e.g., monitoring disease progression in a subject. The methods include obtaining the biological sample from the subject; conducting the methods of analyzing protein composition from individual extracellular vesicles (EVs) from a biological sample as described herein; obtaining sequencing results; and analyzing the sequencing results to determine if the subject has a disease.

In some embodiments, the methods of detecting and monitoring disease progression in the subject include diagnosing the subject with the disease, determining a treatment regimen, monitoring the efficacy of the treatment regimen, and/or determining whether symptoms of the disease in the subject are improving. For instance, the disease can be a cancer, an inflammatory disorder, an immune disorder, a cardiovascular disorder, or a brain-related disorder, such as brain trauma.

In another aspect, the disclosure provides kits for analyzing protein composition from individual extracellular vesicles (EVs) from a biological sample. The kits include antibody-DNA conjugates, each including an antibody that binds to EVs, a T7 promoter, a first barcode region, and a first hybridization region; and barcoded beads, each including a unique molecular identifier (UMI) region, a second barcode region, and a second hybridization region that can hybridize to the first hybridization region.

In some embodiments, the kits also include an extension reagent mix that includes deoxynucleotide triphosphates, a nonionic surfactant, a redox reagent, a DNA polymerase, and a Uracil-Specific Excision Reagent (USER) enzyme. In some instances, the kits also include antibodies that specifically bind to surface antigens present on tumor cells.

As used herein, the term “patient” or “subject” refers to members of the animal kingdom including, but not limited to, mammals, such as, human beings. The term “mammal” refers to all mammals, including, but not limited to human beings.

As used herein, the term “biological sample” or “sample” refers to a sample obtained from a laboratory, such as cultured cells. “Biological sample” or “sample” can also refer to a fluid or tissue obtained from a patient. For instance, any bodily fluid, e.g., blood, urine, saliva, cerebrospinal fluid (CSF), cyst fluids, or fluid obtained from a lavage. Alternatively, a biological sample can be obtained from tissues or organs.

As used herein, the phrase “detecting and monitoring disease progression” refers to: (i) identifying and diagnosing a specific disease in a subject, (ii) identifying a suitable treatment regimen, and (iii) monitoring disease progression, which includes determining if the treatment regimen is achieving a desirable clinical/medical end-point, such as reducing symptoms of the disease; inhibiting the disease, i.e., arresting its development; or relieving the disease, i.e., causing regression of the disease.

As used herein, the term “treatment” or “treating” a disease means administration to a patient by any suitable dosage regimen, procedure, and/or administration route of a composition, device, or structure with the object of achieving a desirable clinical/medical end-point.

As used herein, the term “treatment regimen” refers to any structured and suitable treatment plan designed to improve and maintain the health of a patient suffering from a disease. This can include both drug and non-drug treatment plans.

As used herein, the phrase “efficacy of the treatment regimen” refers both to the maximum response achievable from a specific treatment regimen (e.g., the maximum response achievable from a pharmaceutical drug), and also the capacity to achieve a therapeutic effect or beneficial change from the specific regimen.

As used herein, the phrase “therapeutic effect” is achieved when a desirable clinical/medical end-point has been detected.

As used herein, the phrase “diseased” or “disease-derived” or “disorder” refers to anything that is not normal. For instance, “diseased cells” refer to cells that function in an abnormal way. Likewise, “disease-derived extracellular vesicles” or “disorder-derived extracellular vesicles” refer to extracellular vesicles derived (or shed) from abnormal cells.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawings will be provided by the Office upon request and payment of the necessary fee.

FIG. 1A is a schematic that shows that EVs are labeled with Ab-DNA conjugates and encapsulated with barcoded beads in droplets.

FIG. 1B is a schematic that shows that barcoded beads contain three barcode regions (EV barcode), UMI, and hybridization region (a). The Ab-DNA conjugate has T7 promoter sequences, barcode region for different antibodies, and hybridization region (a*).

FIG. 1C is a microscope image of a droplet generator system in which closely packed barcoded hydrogel beads, labeled EV, extension reagent mix, and oil are introduced through separate input channels to form oil droplets that contain, on average, no more than one labeled EV and no more than one barcoded bead per droplet.

FIG. 2A is a schematic that shows nucleic acid extension in droplets makes a product that includes both the EV barcode and the antibody barcode.

FIG. 2B is a schematic that shows IVT is performed using a T7 promoter sequence.

FIG. 2C is a schematic that shows the products are treated with DNase to remove remaining primers to minimize crosstalk and that RNA products are purified. Purified products are converted to cDNA and amplified using PCR for sequencing.

FIG. 3A is a qPCR graph.

FIG. 3B is an image of a gel electrophoresis showing length of amplicon obtained from qPCR.

FIG. 3C is sequencing data confirming single EV amplicon made using droplets. Sequences shown (from top to bottom) are:

SEQ ID NO: 1:
(T7 product: GGGAGATGGAGGGTGTGTAGTaccgttTCACCATACA
TCTTCACTCACATTCTCNNNNNNaccgagtgatCACAATCACCATACCTt
cgtttctatTCTCTACACCTACCAcatgtacatcTAT CTC CAC ACA
TCC TCA ACC ATC ACT CAC),
SEQ ID NO: 2:
(1-My_Primer-2: GGA GTG GAG GGT GTG TAG),
and
SEQ ID NO: 3:
(2-My_Primer-2: GTGAGTGATGGTTGAGGATGTGTGGAG).

FIG. 4A is a graph showing crosstalk of reads based on sequencing.

FIGS. 4B-1 and 4B-2 are a pair of graphs comparing number of reads obtained from isotype control antibody labeled EV to that from target specific antibody labeled EV.

FIG. 5A is a graph showing profiling results for EVs labeled with EGFR and PD-L1.

FIG. 5B is a map showing sequencing results for 3900 A431 EVs profiled individually for two protein markers, PD-L1 and EGFR

### DETAILED DESCRIPTION

The disclosure provides methods for droplet-based single EV profiling, uses of these methods, and kits for the use of these methods. Specifically, this disclosure is based, at least in part, on the discovery that droplet-based single EV profiling permits the detection and identification of diseased EV subtypes, which would otherwise be impossible to detect due to the co-presence of abundant normal EVs.

To resolve the heterogeneity and rarity of EVs, this disclosure provides a droplet-based single EV protein sequencing platform that overcomes limitations of current bulk measurement technologies [Shao, H., Chung, J., Lee, K., Balaj, L., Min, C., Carter, B S., Hochberg, F H., Breakefield, X O., Lee, H., Weissleder, R. (2015). Chip-based analysis of exosomal mRNA mediating drug resistance in glioblastoma. Nature Communications. 6:6999; Ko, J., Hemphill, M., Yang, Z., Sewell, E., Na, Y J., Sandsmark, D K., Fisher, S A., Torre, E A., Diaz-Arrastia, R., Kim, J., Meaney, D F., Issadore, D. (2018). Diagnosis of traumatic brain injury using miRNA signatures in nanomagnetically isolated brain-derived extracellular vesicles. Lab on a Chip. 18(23), 3617-3630], which make it difficult to discover a rare EV population in the presence of high background. The new technology can profile EV proteins at a single EV level and it enables discovery of different EV subtypes derived from heterogeneous cell populations by identifying each of them. The host cell EV mapping using the technology will open up the opportunity to better reflect molecular changes in tumor immune microenvironment, as well as other diseases. It will also enable the easy monitoring and predicting of patient outcomes to achieve the best possible treatment decisions.

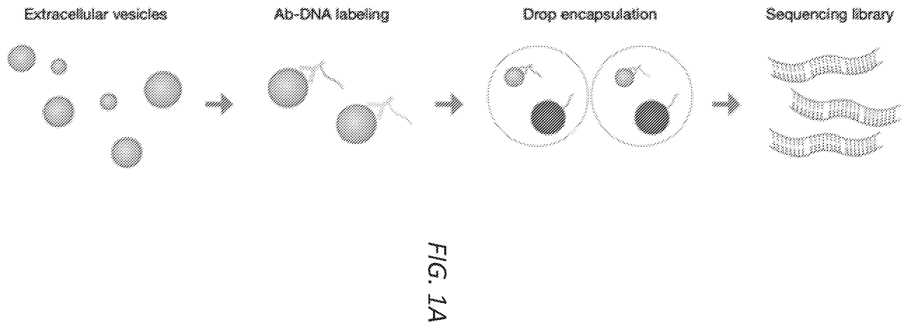

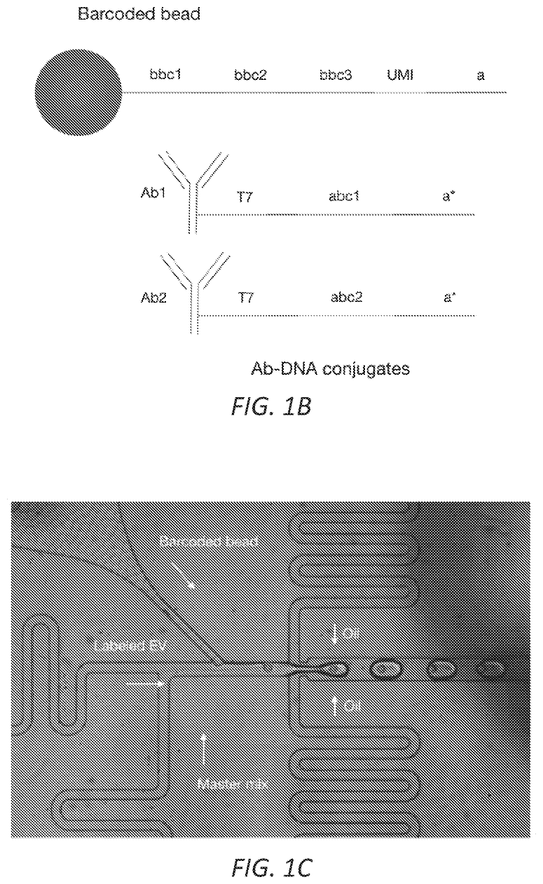

FIG. 1A is a schematic showing isolated extracellular vesicles are labeled with Ab-DNA conjugates of interest and remaining unlabeled Ab-DNA conjugates are washed away, for example, by using a size exclusion chromatography column (not shown). Once the labeled EVs are prepared, they are diluted based on Poisson's ratio to allow single vesicle (1-1000 nm) encapsulation into droplets (diameters (d)=200 μm, water in oil) all of which also include a single barcode bead. Subsequent steps (extension, in vitro transcription, reverse transcription (RT), PCR) are performed to prepare sequencing library. FIG. 1B shows barcoded beads include the bead (d=40 μm acrylamide hydrogel beads), a multi-barcode region (containing bc1, bc2, bc3, . . . , bcn with multi-step extension), a unique molecular identifier (UMI) sequence, and a hybridization region (a) for the capture of the ab-DNA conjugate. FIG. 1B also shows ab-DNA conjugates include an antibody; and a DNA sequence containing the T7 promoter sequence, an antibody barcode (different from the bead barcodes), and a hybridization region (a*) that is complementary to that of the barcoded beads (a). FIG. 1C depicts droplet generator microfluidics showing the encapsulation of single EV and single barcoded beads into each droplet. There are three input channels (barcoded beads, labeled EV, and an extension reagent mix) that make water-in-oil droplets by pinching off the liquid with the oil input.

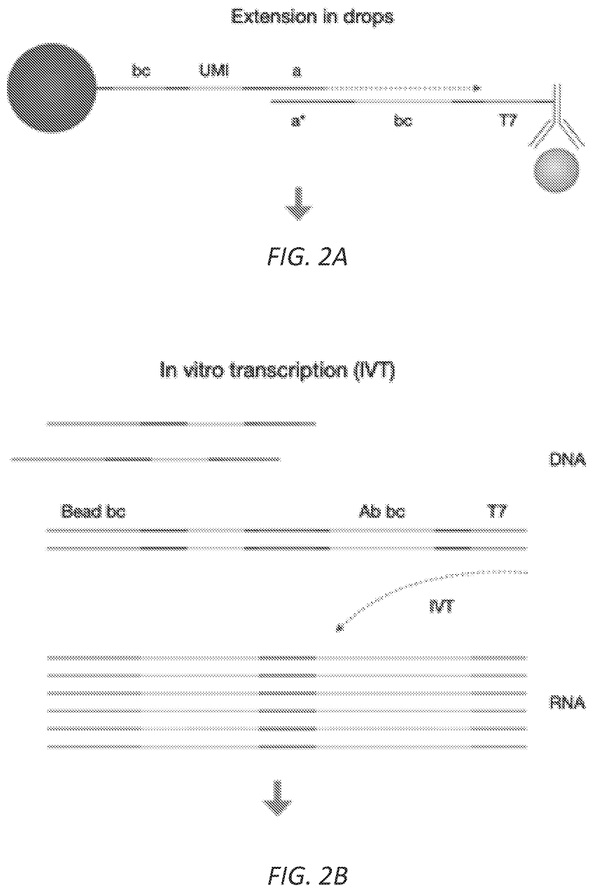

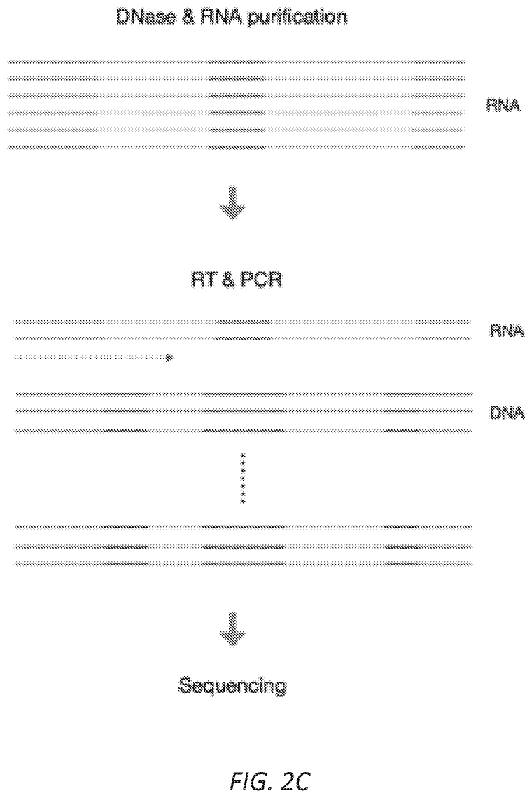

There are two main challenges to current methods of single EV protein profiling. First, the short length (63 bases) of DNA sequences that are used for antibody barcodes makes it difficult for them to be purified from other unwanted primers after extension. Second, single EVs contain much less content (>1000×) than that from single cells due to their small sizes (30 nm-1 μm). To address these challenges, we incorporated IVT, which removes both unwanted DNA primers and amplifies the signal from each EV. FIG. 2A shows, in droplets, the hybridization regions from the barcoded beads (a) and the ab-DNA conjugates (a*) bind to each other and the hybridized product (dotted line) is extended. FIG. 2B shows generation of many RNA copies from a single DNA product is possible by using the T7 promotor sequence that is present in the ab-DNA conjugates (“In vitro transcription,” IVT). FIG. 2C shows following in vitro transcription, the product is treated with DNase to remove any potential crosstalk DNA products and to purify RNA. Reverse transcription is then performed to make cDNA from the RNA product and PCR to amplify the product and prepare for next generation sequencing.

Methods of Sequencing Nucleic Acids from Individual EVs

Extracellular vesicles (EVs) are a family of membrane vesicles containing a phospholipid bilayer that are secreted in the extracellular environment by most cells. They are typically <1000 nm in size and vary in size, molecular composition, biogenesis, and function. The term “extracellular vesicles” encompasses exosomes, microvesicles, exomeres, apoptotic bodies, or oncosomes, as well as other vesicles, like endosomes, lysosomes, or mitochondria. Further, EVs can be extracellular (shed) or intracellular. They can be semi-permeabilized to release intravesicular proteins. They can also be non-circulating or circulating in plasma or serum.

EV-mediated cell-to-cell communication in cancer has been highlighted in recent years, where transfer of EVs from the tumor to the tumor-microenvironment promotes angiogenesis, matrix remodeling and modulating immune and therapy response. Conversely, the transfer of EVs from the tumor microenvironment to tumor cells has been shown to promote tumorigenesis by increasing tumor cell proliferation, migration, epithelial to mesenchymal transition, and resistance to chemotherapy.

EVs are therefore a good source for biomarkers for disease elsewhere in the body, as they reflect the cell of origin in terms of proteins, nucleic acids (mRNA and the variety of smaller non-coding RNAs) and lipids. However, EV analysis is severely hampered by the EV heterogeneity and the complex nature of biological and clinical EV samples.

The family of EVs secreted by a single cell type can be separated into three major classes based on their biogenesis: exosomes, microvesicles, and apoptotic bodies. Exosomes are small vesicles with a diameter in the range between 40 to 100 nm. They are formed within endosomal compartments and secreted by the fusion of multivesicular bodies with the plasma membrane. Microvesicles are generally larger (100-1000 nm) and are formed by direct budding of the plasma membrane. Apoptotic bodies are released upon programmed cell death by membrane blebbing and can be from 50 nm up to 5 μm in diameter. However, due to a significant overlap in size, similarities in composition and lack of specific markers, it is very difficult to assign individual EVs to one of the biogenesis pathways. Moreover, EV are not only shed by diseased/abnormal cells, but they are also shed by normal host cells. As such, differentiating between normal and abnormal EVs is difficult.

Although there are currently available and clinically viable diagnostics of EVs, they are all based on “bulk measurements” requiring 10⁵-10⁶ EV per biomarker to measure protein (e.g., Western, ELISA) or 10²-10³ EV for the more sensitive methods (μNMR, nPLEX). Further, because EVs are also shed by normal cells, current assays are invariably “contaminated” and unsuited for precise analyses of single EVs. Because of the unmet need for single EV analysis, there has been recent interest in developing single vesicle analytical methods. However, multiplexed protein analysis in individual EVs has been difficult.

Description of the Assay Method Steps

The assay methods start with the isolation of EVs from a sample up to and including the analysis of sequencing data. There are 9 main assay steps, some of which are optional, though generally provide better results:

EV Isolation

As shown in FIG. 1A, EVs are first isolated from a sample. EVs can be isolated from a sample by any suitable means that are well-known and routine in the art. This can include, but is not limited to, the following methods.

Ultracentrifugation: Ultracentrifugation utilizes the separation of particles according to their buoyant density by centrifugation.

Filtration: EVs can be separated based on their size, through filtration means (for instance, ultrafiltration, hydrostatic filtration dialysis, gel filtration, and size exclusion chromatography).

Separation based on solubility or aggregation: EVs can be isolated based on EV solubility in super-hydrophilic polymers, such as polyethylene glycols. The procedure reduces to mixing of the sample and polymer solution, incubation, and sedimentation of EVs by low-speed centrifugation. EVs can be isolated by aggregation. Since EVs are negatively charged, protamine, a positively charged molecule, can be used to aggregate and isolate EVs from a sample.

Isolation based on affinity interactions: Lipids, proteins, and polysaccharides are exposed on the surface of EVs. All these substances are potential ligands for manifold molecules, including antibodies, lectins, and lipid-binding proteins. Therefore, the use of molecules specifically interacting with the molecules on the EV outer surface can also be used as a means for EV isolation.

EV Characterization (Optional)

Characterization includes measuring/identifying proteins specific to EV to confirm EV origin (e.g., through immunoblotting), identifying EV structure (e.g., through use of transmission electron microscopy), or quantification of the number of EVs in a sample volume and their size distribution (e.g., through nanoparticle tracking analysis).

EVs are then Labeled with an Antibody-DNA Conjugate

As shown in FIG. 1A, EV's are labeled with an antibody-DNA conjugate. To enable single cell protein profiling, antibodies tagged with known DNA sequences are used. In some aspects, multiple antibody-barcode conjugates can be used, each with different barcodes (e.g., abc1, abc2, abc3, . . . abcn and n=1-1000). To conjugate known DNA tags to antibodies, any suitable means for crosslinking the antibody to the DNA can be used. For instance, by adding bifunctional crosslinkers reactive towards thiol (via maleimide) and amine (via NHS) moieties on the DNA oligos. For instance, by utilizing TCO-PEG4-NHS Ester-click chemistry or DBCO-PEG5-NHS-azide chemistry.

Any antibody that targets a specific surface EV protein of interest can be used. For instance, in immunotherapy monitoring, any antibody that specifically binds to immune markers of the tumor immune microenvironment are suitable. For instance, CD30, FoxP3, CCR8, SiglecF, Ly6G, CCL3, and gal3 can be used in the methods described herein.

As shown in FIG. 1B, the DNA oligos include a T7 promoter sequence, a barcode sequence to distinguish different antibodies, and a hybridization region. Each of the regions can include, as described below anywhere between 5-100 nucleotide bases. T7 promoter sequences are well known and enable in-vitro transcription, as described below. The hybridization region includes complementary sequence to the sequence of the barcoded beads, and can include anywhere between 5-100 nucleotide bases, preferably, 15-25 nucleotide bases, for instance, 100, 75, 50, 30, 25, 20, 25, 10, 9, 8, 7, 6, 5, or anything in between. With respect to the barcode region, each antibody gets its own barcode sequence so that each EV gets its own unique “identity.” This type of barcoding permits the ability to tell which sequences came from which EVs once sequencing is done. The barcode DNA sequence can include anywhere between 5-50 nucleotide bases, preferably 4-6, for instance, 30, 20, 10, 9, 8, 7, 6, 5, or anything in between.

Once the EVs have been labelled with the antibody-DNA conjugate, labelled EVs are purified to remove unbound antibody-DNA conjugates. This can be done by size-exclusion chromatography or any other suitable means.

Barcoded Beads are Prepared

Barcoded beads include a barcode region (which can include multiple regions, e.g., bc1, bc2, bc3, and so on, generated through extension), a unique molecular region, and a hybridization region, as shown in FIG. 1B. The barcode region is synthesized using a 3-step extension and can include 5-50 nucleotide bases, for instance, 50, 30, 20, 10, 9, 8, 7, 6, 5, or anything in between. The barcodes are extended three times with 96 primer diversity each time to achieve high throughput EV profiling. In some aspects, libraries of synthetic barcodes are designed and generated in such a way that i) they are different from each other in at least one base, ii) each have a length of 5-20 bases, and iii) are non-human origin. The unique molecular identifier (UMI) sequence is present to correct for any bias and provide accurate quantitative results. The UMI can be anywhere between 5-50 nucleotide bases, preferably 6-10 nucleotide bases, for instance, 30, 20, 10, 9, 8, 7, 6, 5, or anything in between. The hybridization region includes complementary sequence to the sequence of the DNA on the antibody-DNA conjugate, and can include anywhere between 5-50 nucleotide bases, preferably 15-25 nucleotide bases, for instance, 30, 20, 10, 9, 8, 7, 6, or 5, or anything in between. Generally, beads can be made from polyacrylamide, cross-linked polyacrylamide, polymers that are easily dissolvable on demand, sepharose, or hydrogels. Alternatively, the beads can be made using Drop-seq beads, inDrops beads, or 10× genomics beads. For instance, Acrydite™ DNA is used to make acrylamide-based hydrogel beads.

Labeled EVs and Barcoded Beads are Encapsulated

Once the EVs have been labelled with the antibody-DNA conjugate and the barcoded beads, the EVs and beads are encapsulated into droplets, as shown in FIGS. 1A and 1C. For example, by a droplet making instrument, e.g., a droplet generator such as the Biorad Automated Droplet Generator, which generates 20,000 droplets per sample. Each droplet, in addition to containing the EV and barcoded bead, contains an extension reagent mix of deoxynucleotide triphosphates (dNTPs), a nonionic surfactant (e.g., Triton X), redox reagent (e.g., dithiothreitol), DNA polymerase (e.g., Taq DNA polymerase; BST 2.0 warmstart), and Uracil-Specific Excision Reagent (USER) enzyme.

Microfluidic encapsulation systems are known in the art and any suitable means for encapsulation can be used. Detailed descriptions of how these systems work are provided, for instance, in U.S. Pat. No. 9,068,181, which is incorporated herein by reference in its entirety. Briefly, particles (e.g., EVs, barcoded beads or other particles), in a fluid stream can be encapsulated in individual droplets by first forming an ordered stream of particles in the fluid stream within a microchannel and then forming the fluid stream containing the ordered stream of particles into droplets each containing, on average, a single particle. For example, a fluid stream entering a droplet forming nozzle can contain two evenly-spaced streams of particles (e.g., EVs) whose longitudinal order is shifted by half the particle-particle spacing. Ordering of the particle within the fluid stream can occur when a high density suspension of particles (e.g., cells or particles) is forced to travel rapidly through a high aspect-ratio microchannel, where particle diameter is a large fraction (e.g., 10-40%) of the channel's narrowest cross-sectional dimension (i.e., a microfluidic channel having at least one cross-sectional dimension that is about 2.5 to about 10 times the width of the largest dimension of the particles). This phenomenon provides a method to controllably load single-EVs into droplets, overcoming the intrinsic limitations set by Poisson statistics and ensuring that a high percentage (e.g., 90% or more) of the droplets contains exactly one EV. Further, multiple microchannels and various microchannel configurations can be used such that each droplet contains exactly one EV and exactly one barcoded bead.

Extension

Inside the droplet, the bead hybridization region hybridizes with the hybridization region on the antibody-DNA conjugate. Beads are collected and extension (i.e., step wherein DNA polymerase adds nucleotides) is performed to create a double-stranded DNA that consists of both the sequence coming from the barcoded bead and the sequencing coming from the antibody-DNA conjugates, as shown in FIG. 2A.

Droplets are Broken (optional)

After extension, the droplets are broken by any suitable means, such as by using perfluorooctanol (PFO), or by applying an electric field to break the droplets. In some embodiments, this step is optional and the droplets are not broken.

In Vitro Transcription

Next, in vitro transcription (IVT) is performed on the DNA strands, as shown in FIG. 2B. IVT allows for template-directed synthesis of RNA molecules of any sequence from short oligonucleotides to those of several kilobases in μg to mg quantities. As such, IVT serves two main purposes: first, thousands of RNA can be made from a single DNA strand, which allows signal amplification that is crucial to profile single EV. Second, by generating RNA from DNA, it is then possible to remove all the DNAs that include dimers and unnecessary products that can cause crosstalk between droplets.

Sequencing

After IVT, as shown in FIG. 2C, RNA is purified, converted to cDNA, amplified by PCR, and then sequenced. cDNA is made by RT-PCR Reverse Transcription (RT)-PCR refers to the use of reverse transcription to generate a complementary cDNA molecule from an RNA template, thereby enabling the DNA polymerase chain reaction to operate on RNA. Polymerase chain reaction (PCR) is a process of amplification of known DNA fragments by serial annealing and re-annealing of small oligonucleotide primers, resulting in a detectable molecular signal. Sequencing (Sanger sequencing, next generation sequencing, or any other suitable means to determine nucleic acid sequence) is well known in the art, and is performed subsequently.

### Methods of Use and Applications of the Droplet-Based Single EV Sequencing Methods

Detecting Disease

Circulating extracellular vesicles (EV) are typically <1,000 nm in size, occur at concentrations of up to 10⁹-10¹¹ vesicles/ml of peripheral blood in patients, are fairly stable over time [Nilsson et al., 2009, #24094] and have been shown to contain small amounts of proteins and even nucleic acids reflective of those found in parental cells [Graner et al., 2009, #98403; Mathivanan et al., 2010, #86387]. The vesicles differ in size, molecular composition, biogenesis and function [Théry, 2015, #34192; Colombo et al., 2014, #87619] and include exosomes and microvesicles among other membrane vesicles [Schorey and Harding, 2016, #72941; E L Andaloussi et al., 2013, #15817; Raposo and Stoorvogel, 2013, #81065]. EV are not only shed by tumor cells (tEV) but also by host cells (hEV). Furthermore, bulk EV protein content has been shown to vary temporally [Eitan et al., 2017, Sci Rep, 7, 1342] and recent studies in our group have shed some light on the composition of individual vesicles investigating abundant proteins [Lee et al., 2017, ACS Nano; Fraser et al., 2019, Neuro Oncol, 21, 606-615]. One emerging view is that the protein expression in well-defined vesicle populations (e.g. exosomes only) varies considerably from one vesicle to the next.

A number of different analytical methods have been developed to analyze EV [Shao et al., 2018, Chem Rev, in press; Im et al., 2017, Lab Chip, 17, 2892-2898; Im et al., 2015, Expert Rev Mol Diagn, 15, 725-33], most of them relying on bulk measurements requiring 103-6 EV for a single measurement. Yet, the identification of a small number of tumor originating vesicles (such as those found in early cancers) in a background of host EV may be impossible by bulk methods. One way to solve the problem is to develop single (“digital”) EV analysis (SEA) techniques. Such single EV analysis could be valuable not only for early detection, but also for studying tumor heterogeneity and phenotypic changes occurring during therapy. Because of the unmet need for single vesicle analysis, there has been increasing interest in this challenge. Some recent approaches of single vesicle analyses have included optical trapping [Prada et al., 2016, Biotechniques, 60, 35-41], Raman spectroscopy [Gualerzi et al., 2017, Sci Rep, 7, 9820], flow cytometry [Smith et al., 2015, #42821; Löf et al., 2016, #70670], and cyclic imaging [Lee et al., 2017, ACS Nano]. So far, only the latter method allows rapid multiplexed protein analysis in individual vesicles. One limitation however is that rare proteins are hard to detect by optical sensing.

The present disclosure overcomes the sensitivity limitation by developing sequencing-based single EV protein profiling. The approach utilizes single cell sequencing [Klein et al., 2015, Cell, 161, 1187-1201; Macosko et al., 2015, Cell, 161, 1202-1214; Weissleder and Pittet, 2020, Nat Biomed Eng]. In contradistinction to scRNAseq however, the present disclosure overcomes a number of challenges: i) an average exosome has a 10⁶ times smaller mass compared to a single cell, ii) the new methods can provide protein profiles from single EVs [Gyuris et al., 2019, Cell Rep, 27, 3972-3987.e6].

The new methods also allow one to profile millions of EV and hundreds of markers of interest in one experiment, so that rare EV subtypes (e.g., mutated proteins) could be identified with reasonable certainty. The new methods provide a new pipeline for antibody-based immune-sequencing that is able to result in readouts from single EVs. The methods use droplet microfluidics to encapsulate individual antibody-DNA labeled EV into droplets that contain unique barcoded beads. The methods also include multiple extension and amplification steps.

The early detection of any disease is far more effective in achieving a cure, and as such, is desirable.

In some instances, the disease type is cancer. Molecular signatures that are found at an early stage of cancer are very subtle. However, the body undergoes significant changes even before tumor cells appear. Since the methods described herein are able to separate healthy cells from non-healthy cells, these changes can be detected using the methods described herein.

In another instance, the disease type is a brain-related disease or brain trauma. This includes, those that are neurological, those caused by trauma, or those that are genetic in origin. Cerebral vasculature serves much more than plumbing for the brain; it also forms an interface and a barrier between the brain and the circulating blood. Brain-derived particles such as organelles or extracellular vesicles, e.g., microvesicles (MVs), exosomes, exomeres, oncosomes, and apoptotic bodies can also be shed from brain tissue into the circulation. As a result, EVs can serve as rich diagnostic markers, e.g., “footprints,” of brain disorders and disease, such as concussions and traumatic brain injury of various levels, when the clinical signs are uncertain. The methods described herein can isolate these EVs from a patient's blood and the droplet-based single extracellular vesicle sequencing technology described herein can be used to get molecular information of the brain-related diseases.

In another instance, the disease can be a virus, such as HIV. When HIV is in its latency period, it is hard to detect despite its presence in the body. During this period, a highly sensitive diagnostics that can monitor the viral load is needed. The methods described herein can be used to find a very small subset of EVs that are shed from infected host cells during this period.

In other instances, the disease is an inflammatory disorder, is an immune disorder, or a cardiovascular disorder.

Determining Treatment Regimens and

Monitoring Efficacy of the Treatment Regimens

Just as important as early detection is to select a therapy or therapeutic regimen. The methods described herein use the droplet-based single EV sequencing assay methods to not only provide early detection of a disease type, but in combination with the appropriate reference standards, can be used to determine and compare the predicted efficacy of different therapeutic regimens in a specific patient.

The droplet-based single EV sequencing assay methods described herein can be used for both initial screening and to determine the best therapeutic regimen. The use of the droplet-based single EV sequencing assay assays described herein, in which each EV can give rise to hundreds of molecular signals, dramatically enhances the ability to detect, evaluate, and monitor EVs in patients with a disease, and to quantitatively monitor and analyze their response to therapeutic interventions.

For instance, immunotherapy for cancer has been successful to cure patients who were previously incurable. However, there is a subset of patients who do not respond to specific therapies. It is important to better understand how patients respond to therapy over time, but obtaining tumor cells on a regular basis from a patient is challenging. The methods described herein can be used to obtain EVs from blood that comes from the tumor immune microenvironment (TIME) and can be used to monitor the effectiveness of immunotherapies over time.

### Kits for Droplet-Based Single EV Sequencing

Additionally provided herein, a kit is provided, which includes the necessary reagents needed to accomplish the methods described herein. For instance, provided herein is a kit for analyzing protein composition from individual extracellular vesicles (EVs) from a biological sample comprising: antibody-DNA conjugates each antibody-DNA conjugate comprising an antibody that binds to the EVs, a T7 promoter, a first barcode region, and a first hybridization region; and barcoded beads each comprising a unique molecular identifier (UMI) region, a second barcode region, and a second hybridization region that can hybridize to the first hybridization region.

### EXAMPLES

The invention is further described in the following examples, which do not limit the scope of the invention described in the claims.

Glioblastoma (GBM) is a highly malignant brain tumor with a poor prognosis [Skog, J., Würdinger, T., van Rijn, S., Meijer, D H., Gainche, L., Sena-Esteves, M., Curry, W T., Carter, B S., Krichevsky, A M., Breakefield, X O. (2008). Glioblastoma microvesicles transport RNA and proteins that promote tumour growth and provide diagnostic biomarkers. Nature cell biology, 10(12), 1470-6; Ricklefs, Fla., Alayo, Q., Krenzlin, H., Mahmoud, A B., Speranza, M C., Nakashima, H., Haves, J L., Lee, K, Balaj, L., Passaro, C., Rooj, A K, Krasemann, S., Carter, B S., Chen, C C., Steed, T., Treiber, J., Rodig, S., Yang, Nakano, I., Lee, H., Weissleder, R, Breakefield, X O., Godlewski, J., Westphal, M., Lamszus, K., Freeman, G J., Bronisz, A., Lawler, S E., Chiocca, E A. (2018). Immune evasion mediated by PD-L1 on glioblastoma-derived extracellular vesicles. Science advances, 4(3), eaar2766]. Immune evasion is mediated by PD-L1 on glioblastoma-derived extracellular vesicles [Science advances, 4(3), eaar2766; Stupp, R, Mason, W P., van den Bent, M J., Weller, M., Fisher, B., Taphoorn, M J B., Belanger, K., Brandes, A A., Marosi, C., Bogdahn, U., Curschmann, J., Janzer, R C., Ludwin, S K., Gorlia, T., Allgeier, A., Lacombe, D., Cairncross, J G., Eisenhauer, E., Mirimanoff, R O. (2005). Radiotherapy plus concomitant and adjuvant temozolomide for glioblastoma. New England Journal of Medicine. 352, 987-996]. Extracellular vesicles (EVs) derived from tumor-infiltrating immune cells can potentially be used as a minimally invasive marker to monitor treatments. However, these EVs are usually hard to detect due to their scarcity compared to the background of healthy cell derived EVs. To solve these currently intractable problems, the present disclosure provides a highly sensitive molecular diagnostic tool that can detect the molecular changes during the course of treatments. The single cell sequencing technique was modified to enable signal detection from single extracellular vesicles, which are 100 times smaller than a cell. Using this ultra-high sensitive technology, millions of extracellular vesicles can be profiled at a single vesicle level and measure expressions levels of hundreds of markers. This highly multiplexed and sensitive technology opens the door to new rare subtype discovery that can be helpful for early glioblastoma diagnostics and beyond.

### Materials and Methods (Used for all Examples Described Below)

Cell Culture for EV Isolation

Glioblastoma Gli36 wt and gli36vIII cell lines were used to test and optimize the seSEQ technology. Cells were grown in a 150 mm cell culture dish and expanded to 8-12 dishes for EV collection. Cells were grown and passaged in DMEM (10% FBS, 1% Penicillin/Streptomycin). Once confluent, media was changed to exosome-depleted DMEM (5% exosome-depleted FBS, 1% Penicillin/Streptomycin) and supernatant was collected 48 hours after the media change. The collected supernatant was spun at 400 g for 5 minutes and filtered with a 0.22 μm vacuum filter to remove any cellular debris.

EV Isolation (Ultracentrifugation, Size Exclusion Column)

For ultracentrifugation, cell cultured supernatant was centrifuged (Beckman Coulter) at 100,000 g for 70 minutes at 4° C. two times. The EV pellet was resuspended in PBS and aliquoted and stored in

−80° C. until usage. For size exclusion chromatography column, mouse plasma was loaded on the qEV column (Izon science) and the protocol from the company was followed to collect EVs from the sample.

EV Characterization (Qubit, NTA)

After EV isolation, EVs were characterized in two different ways. The protein concentration was measured using Qubit (Thermo Fisher) and the particle number was calculated using nanoparticle tracking analysis (NTA). For Qubit, the protein assay kit (Thermo Fisher) was used and the company protocol was followed for measurement. For NTA, the measurement was done at the Nanosight Nanoparticle Sizing and Quantification Facility at MGH. Three 30 sec measurements were performed and averaged from each sample. The same parameters were used for analysis (Image: Screen gain of 7.4, Camera level of 11, Detection: Screen gain of 10, Detection threshold of 13).

Antibodies

Cetuximab (anti-EGFR antibody, Erbitux) and anti-CD63 antibody (Ancell, 215-820) were used to test and optimize the technology. All antibodies were checked for the absence of bovine serum albumin (BSA) for Ab-DNA conjugation. All antibodies were tested on positive cell lines and validated before usage.

DNA Barcodes

Two DNA barcodes were used in this study. First, DNA barcodes for beads were synthesized using a 3-step extension. Acrydite™ DNA was used to make acrylamide-based hydrogel beads and barcodes were extended for three times with 96 primer diversity each time to achieve high throughput EV profiling. Second, the DNA for antibodies consisted of three regions: T7 promoter sequences for in vitro transcription (IVT), barcode sequence, and a universal sequence complementary to the sequence of barcoded beads (also referred to as hybridization sequence).

Ab-DNA Conjugation

BSA free antibodies were buffer exchanged to biocarbonate buffer (pH 8.4) using a 40 k Zeba column (Thermo Fisher, 87765). The antibody was incubated with TCO-PEG4-NHS Ester (Click Chemistry Tools, A137-10) for 25 mins at room temperature and unlabeled TCO-PEG4-NHS Ester was removed using a 40 k Zeba column. Degree of labeling (DOL) was checked by incubating antibodies with Cy3 Tetrazine (Click Chemistry Tools, 1018-1) for 25 mins at room temperature and remaining Cy3 Tetrazine was removed using a 40 k Zeba column. Cy3:Antibody ratio was measured using the Nanodrop UV/Vis mode (Thermo Scientific) at A550/A280. 1 mM of amine-modified DNA oligo (Integrated DNA Technologies) was buffer exchanged to borate buffer (pH8.5) using a 7 k Zeba column (Thermo Fisher, 89878).

The DNA oligo was incubated with Methyltetrazine-PEG4-NHS Ester (Click Chemistry Tools, 1069-10) for 25 mins at room temperature and unlabeled Tz-PEG4-NHS was removed using three 7 k Zeba columns. Tz:DNA ratio was measured using the Nanodrop UV/Vis mode at A520/A260. TCO labelled antibody and Tz labelled DNA were mixed with appropriated DNA excess (Cy3:Antibody ratio—0.5) and incubated for 45 mins at room temperature. The conjugation was validated using the NuPAGE 4-12% Bis-Tris Protein Gel (Thermo Fisher, NP0321BOX). Unconjugated antibody and DNA-conjugated antibody were incubated with 4× NuPAGE LDS Sample Buffer (Thermo Fisher, NP0007) for 5 mins at 75° C. and loaded on the gel with Novex Sharp Pre-stained Protein Standard (Thermo Fisher, LC5800). The gel was run in 20× NuPAGE MOPS SDS Running Buffer (Thermo Fisher, NP0001) for 1 hour at 120V. The validated Antibody-DNA conjugate was stored in 4° C. until usage.

EV Labeling and Purification

EV was labeled with 10 μg/ml of Ab-DNA conjugates in 1% BSA-PBS for 1 hour and purified using size exclusion chromatography qEV column (Izon science) to remove unlabeled Ab-DNA conjugates. A single use qEV column was used and 400 μl was collected after dead volume to achieve a pure EV population. The labeled EV was stored in 4° C. until usage and used within a few days to prevent degradation.

Barcoded Bead Fabrication

A 500 μL solution mix was prepared containing 50 μL TBSET buffer, 30 μL 10% (w/v) APS (Sigma-Aldrich, A9164), 75 μL 40% (v/v) Acrylamide solution (Sigma-Aldrich, A4058-100ML), 20 μL 250 μM Acrydite™-modified DNA primers (obtained from IDT; SEQ ID NO: 4: ATTATATATATU GTGAGTGATGGTTGAGGATGTGTGGAG), 245 μL 0.8% (w/v) BAC (Sigma-Aldrich, A4929-5G) and 80 μL H₂O. This solution was loaded into a 1-mL syringe (Becton Dickinson, 309628). 1.5 mL carrier oil (RAN Biotechnologies, 008-FluoroSurfactant-2wtH-50G) and 6 μL of TEMED (Sigma-Aldrich, T9281-25ML) were mixed and loaded into a 3-mL syringe (Becton Dickinson, 309657). These two syringes were connected to inlets of the droplet generation device by PE2 tubing (Scientific Commodities, BB31695-PE/2). The aqueous solution was pumped at 500 μL/hour and the oil was pumped at 1000 μL/hour. The emulsion droplets were collected at the outlet of the microfluidics chip.

The collected droplets were covered with 200 μL mineral oil (Sigma-Aldrich, M5310-4L) and incubated at 70° C. overnight. Subsequently, the droplets were centrifuged and the carrier oil phase and mineral oil phase were discarded. 500 μL 20% (vol/vol) PFO (Alfa Aesar, B20156) in HFE 7500 (Novec 7500) was used to break the drops. The beads in the aqueous phase were washed with 1% Span-80 (Sigma-Aldrich, S6760-250ML) in hexane (Sigma-Aldrich, 227064-1L) twice and then with TBSET buffer 3 times. Beads were then filtered through a 70 μm cell strainer (Corning, 352350) and were then stored in TET buffer at 4° C. for up to 6 months.

Device Fabrication

The microfluidic device for droplet generation was fabricated at the Soft Materials Cleanroom (SMCR), Harvard Center for Nanoscale Systems (CNS). The device (height (h)=100 μm) was made using soft lithography with SU-8 3050. The PDMS that consists of microfluidic channels were bonded with glass using plasma bonding. The device was made hydrophobic before usage by treatment with 1% Trichloro(1H,1H,2H,2H-perfluorooctyl)silane in Novec 7500 (Oakwood Chemical).

Imaging and Image Analysis

Zeiss Axio Observer Z1 inverted fluorescence microscope (Wyss Institute) was used to acquire fluorescent images. GFP, Cy3, and Cy5 filter cubes were used to excite FAM, HEX, and Cy5 fluorophores respectively. For imaging analysis, ImageJ and CellProfiler were used to measure fluorescent intensity and to count droplets.

seSEQ Protocol

EVs were first isolated from plasma or cell cultured media using ultracentrifugation or size exclusion chromatography. Isolated EVs were labeled with antibody-DNA conjugates and purified using size-exclusion chromatography to remove unbound antibody-DNA conjugates. Labeled EVs were then encapsulated into droplets (one EV per droplet using Poisson distribution) along with barcoded beads and an extension reagent mix (19.2 μl 10 mM dNTP, 6.48 μl 10% triton, 14.4 μl 100 mM DTT, 14.4 μl 10×TP, 5.76 μl BST 2.0 warmstart, and 4.32 μl USER enzyme). Within the collected droplets, an extension step was performed (60° C. for 2 hr) using a thermal cycler. The droplets were then broken using PFO and the upper phase was collected for IVT using MEGAshortscript T7 Transcription kit (Thermo Fisher). We then purified RNA using an AMPure bead (Beckman Coulter) with 1.6× volume of the sample and eluted the sample in RNA elution buffer. We performed reverse transcription (RT) using Maxima H Minuse Reverse Transcriptase (Thermo Fisher). After RT, we performed PCR and prepared for sequencing. Sequencing was performed using a Next Gen Sequencing service from the Genewiz company.

### Example 1: Droplet Microfluidic Platform for Single EV Immuno-Sequencing

Target-specific antibodies of interest were conjugated to DNA sequences that serve as a unique barcode. Specifically, we used the bio-orthogonal trans-cycloctene/tetrazine (TCO/Tz) click chemistry to rapidly and efficiently conjugate Ab-DNA at high yields [Ko et al., Advanced Biosystems, 1900307]. The DNA barcode in each antibody consisted of three sequence regions: a complementary sequence to bind to unique beads in the droplet, the actual Ab defining barcode, and a T7 promoter sequence for in vitro transcription (IVT). Isolated EV were first labeled with Ab-DNA and remaining unbound Ab-DNA was washed using size exclusion chromatography (Izon) [Ko et al., Advanced Biosystems, 1900307] (FIG. 1A).

We used a four-channel droplet microfluidic device for single EV and bead encapsulation in droplets (FIG. 1C). Barcoded beads, labeled EV, oil, and master mix for an extension step are introduced through each channel to form droplets. We closely packed the beads by designing a channel that is narrower (50 μm in width) than the size of the beads (60 μm in diameter), which can achieve efficient single bead loading per droplet [Abate et al., 2009, Lab Chip, 9, 2628-31]. Using this droplet maker, we created 180 μm droplets that contain beads and EV in master mix solution. Single EV encapsulation conditions were explored and validated in our previous study using Poisson distribution with flow rates, EV input concentration, and droplet volume [Ko et al., Advanced Biosystems, 1900307].

After droplet encapsulation, multiple extension and amplification steps are sequentially performed to synthesize amplicons for sequencing. We used a new technique to fabricate dissolvable polyacrylamide beads by crosslinking acrylamide with disulfide bridges that can be cleaved by dithiothreitol (DTT) [Wang et al., 2020, Advanced Science, 1903463]. Once cleaved, these beads rapidly release barcode primers (<3 mins at 1 mM DTT), highly increasing reaction efficiency in droplets and achieving high loading (>95%) of a single bead per droplet. The dissolvable polyacrylamide beads consist of three sequence regions: a complementary sequence to Ab-DNA, unique molecular identifier (UMI), and EV barcodes made using multiple split-pool approaches to increase diversity.

### Example 2: Single EV Immuno-Sequencing Pipeline

The single EV immune-sequencing pipeline included five steps: extension, IVT, purification, reverse transcription (RT), and PCR (FIG. 2). The first step is an extension in droplets (FIG. 2A). During this step, primers from barcoded beads hybridize to Ab-DNA and perform extension to generate a single strand that contains all the necessary information including EV barcode, UMI, and Ab barcode. We incorporated IVT in our pipeline to achieve two goals, i) signal amplification for single EV readout and ii) removal of a potential source of crosstalk (FIG. 2B). Multiple RNA copies were synthesized from Ab-DNA that contains a T7 promoter sequence. After IVT, original DNA template strands and incompletely extended DNA products are removed using DNase to minimize crosstalk (FIG. 2C). Once DNA were removed, amplified RNA are purified using AMPure XP magnetic beads and converted to cDNA using RT. Converted cDNA were amplified using PCR for sequencing library.

### Example 3: Validation of Amplicon Synthesized for Single EV Profiling

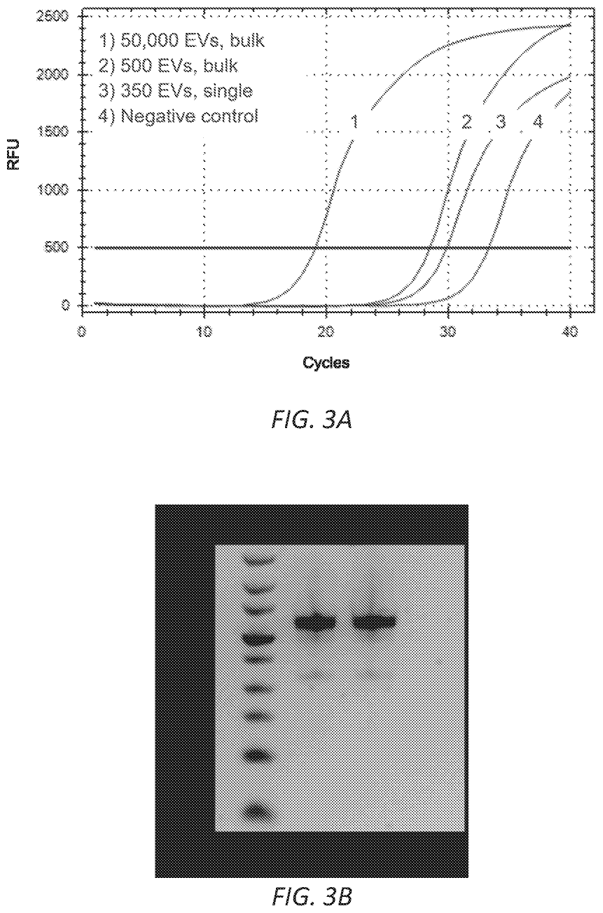

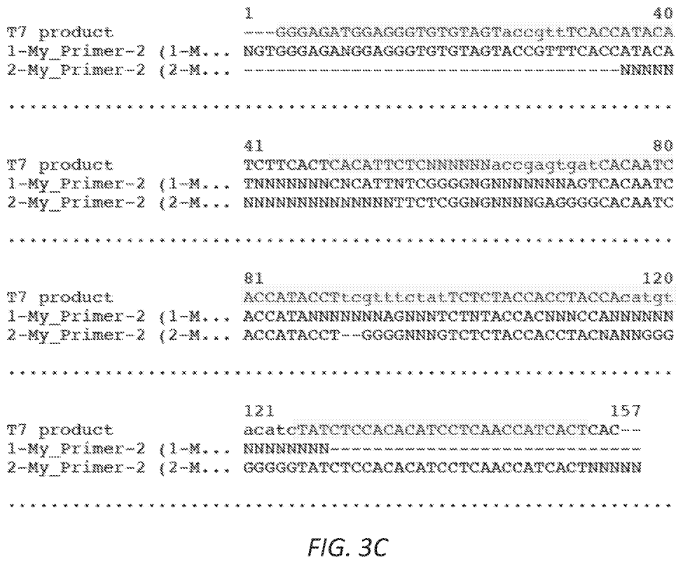

To validate a successful amplicon synthesis, we first performed qPCR with converted cDNA (FIG. 3A). Two positive control samples with a different number of bulk EV were prepared in tubes. For single EV, a total number of 350 EV and a negative control sample without EV were prepared using droplets. Both bulk and single EV samples showed excellent amplifications. The length of the amplicon was checked using a gel where both amplicons made in a tube for bulk EV and using droplets for single EV showed an expected length (150 bp) (FIG. 3B). The single EV amplicon made using droplets was further checked using Sanger sequencing (FIG. 3C). The amplicon sequence matched well to the template sequence, confirming successful amplicon synthesis for single EV protein profiling.

### Example 4: Evaluation of Single EV Sequencing Using Crosstalk and Control Experiments

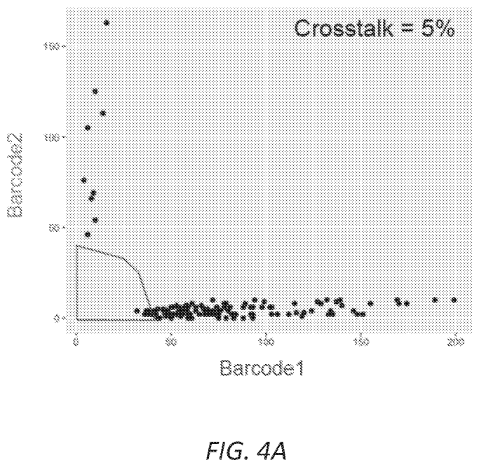

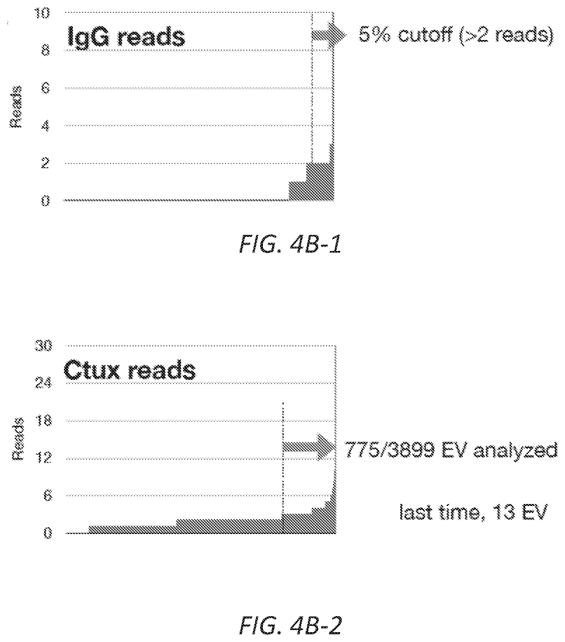

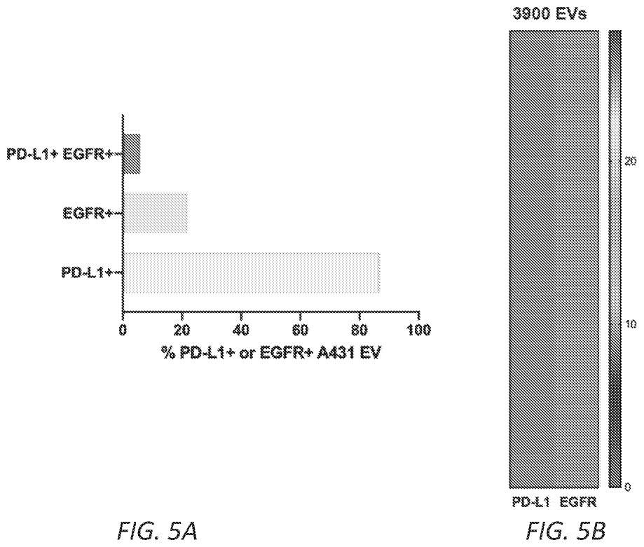

To evaluate the accuracy of the single EV profiling technology, crosstalk of reads was measured using sequencing (FIG. 4A). Two different Ab-DNA were prepared with anti-EGFR antibody conjugated with two different DNA barcode sequences. Both Ab-DNA were used to separately label Gli36-glioma cell line derived EV and the labeled EV were mixed together prior to droplet encapsulation. The developed pipeline was used to synthesize sequencing amplicons and the sequencing data was aligned to each barcode sequence to measure crosstalk reads. A majority of the reads was correctly aligned to one barcode sequence and only 5% of them were aligned to both barcode sequences. This percentage can further be lowered based on the threshold set for data analysis.

To evaluate the specificity of the single EV profiling technology, a control experiment was performed by comparing number of reads obtained from isotype control antibody labeled EV to that from target specific antibody labeled EV (FIG. 4B). Gli36-glioma cell line derived EV were labeled with both anti-IgG isotype control antibody and anti-EGFR antibody and single EV were sequenced using the developed pipeline. Background threshold (2 reads) was determined using a 95% confidence interval of the reads from anti-IgG-DNA. When the threshold was applied to the reads from anti-EGFR-DNA, recovering ˜20% of the total reads. The low percent recovery was expected due to scarce protein contents from individual EV. The ability to sequence single EV at high throughput and small number of reads required for single EV profiling (<100 reads/EV per target) can overcome an inherent challenge of the presence of low signal in single EV.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of analyzing protein composition of individual extracellular vesicles (EVs) from a biological sample, the method comprising
isolating EVs from a biological sample;
labeling the EVs with antibody-DNA conjugates, wherein the antibody-DNA conjugates each comprise (i) an antibody that binds to the EVs, (ii) a T7 promoter region, (iii) a first barcode region, and (iv) a first hybridization region;
obtaining barcoded beads;
encapsulating the labeled EVs, the barcoded beads, and an extension reagent mix into droplets;
within one or more of the droplets, hybridizing a first hybridization region in the antibody-DNA conjugates with a second hybridization region in the barcoded beads to create hybridized DNA;
extending the hybridized DNA within one or more of the droplets to generate extended DNA;
generating RNA from the extended DNA;
synthesizing cDNA from the RNA;
amplifying and sequencing the cDNA from one or more individual EVs from the biological sample; and
analyzing the sequence of the cDNA from individual EVs to define their protein composition.

2. The method of claim 1, wherein the extension reagent mix comprises deoxynucleotide triphosphates, a nonionic surfactant, a redox reagent, a DNA polymerase, and a Uracil-Specific Excision Reagent (USER) enzyme.

3. The method of claim 1, wherein the EVs are isolated from the biological sample by ultracentrifugation or size exclusion chromatography.

4. The method of claim 1, further comprising purifying EVs labeled with antibody-DNA conjugates.

5. (canceled)

6. The method of claim 1, wherein the barcoded beads comprise beads conjugated to a unique molecular identifier (UMI) region, a second barcode region, and the second hybridization region, which can hybridize to the first hybridization region.

7. The method of claim 1, wherein on average, only one labeled EV and only one barcoded bead are encapsulated per droplet.

8. The method of claim 1, wherein RNA is generated from extended DNA by performing in vitro transcription (IVT) on the extended DNA.

9. The method of claim 1, wherein the cDNA is amplified by conducting a polymerase chain reaction (PCR).

10. The method of claim 1, wherein the antibody that binds to the EVs specifically binds to surface antigens present on a tumor cell.

11. The method of claim 1, further comprising characterizing the EVs after isolation from the biological sample.

12. The method of claim 1, wherein the beads comprise one or more of a polyacrylamide, a cross-linked polyacrylamide, a polymer dissolvable on demand, a sepharose, or a hydrogel.

13. (canceled)

14. (canceled)

15. The method of claim 6, wherein each of the UMI region, the second barcode region, and the second hybridization region comprises 5-50 nucleotide bases.

16. The method of claim 6, further comprising generating libraries of synthetic barcodes, wherein each barcode is different from each other barcode in at least one base, and wherein each barcode comprises 5-20 nucleotide bases.

17. (canceled)

18. The method of claim 1, wherein the biological sample is obtained from cultured cells or comprises blood, saliva, urine, cerebrospinal fluid, cyst fluid, or a lavage from a patient.

19. (canceled)

20. The method of claim 1, wherein the EVs are semi-permeabilized to release intravesicular proteins.

21. The method of claim 1, wherein the EVs are microvesicles, exomeres, apoptotic bodies, oncosomes, endosomes, lysosomes, or mitochondria.

22. A method of detecting and monitoring disease progression in a subject, the method comprising
obtaining the biological sample from the subject;
conducting the method of claim 1;
obtaining sequencing results; and
analyzing the sequencing results to determine if the subject has a disease.

23. The method of claim 22, wherein detecting and monitoring disease progression in the subject comprises diagnosing the subject with the disease, determining a treatment regimen, monitoring the efficacy of the treatment regimen, and determining whether symptoms of the disease in the subject are improving.

24. The method of claim 22, wherein the disease is a cancer, an inflammatory disorder, an immune disorder, a cardiovascular disorder, or a brain-related disorder.

25-28. (canceled)

29. The method of claim 1, wherein the beads are dissolvable beads.


SEQUENCE LISTING (4 sequences)

SEQ ID NO: 1 - DNA, 152 bp, Artificial Sequence
  sequencing primer
  misc_feature (55)..(60): n is a, c, g, or t
gggagatggagggtgtgtagtaccgtttcaccatacatcttcactcacattctcnnnnnnaccgagtgatcacaatcaccataccttcgtttctattctctaccacctaccacatgtacatctatctccacacatcctcaaccatcactcac

SEQ ID NO: 2 - DNA, 18 bp, Artificial Sequence
  sequencing primer
ggagtggagggtgtgtag

SEQ ID NO: 3 - DNA, 27 bp, Artificial Sequence
  sequencing primer
gtgagtgatggttgaggatgtgtggag

SEQ ID NO: 4 - DNA, 39 bp, Artificial Sequence
  sequencing primer
attatatatatugtgagtgatggttgaggatgtgtggag